(19) United States
(12) Patent Application Publication
(10) Pub. No.: US 2016/0284036 A1
(43) Pub. Date: Sep. 29, 2016

(54) METHOD, APPARATUS, AND COMPUTER PROGRAM PRODUCT FOR DYNAMIC EVALUATION OF CONSUMABLE CONSUMPTION

(71) Applicant: McKesson Corporation, San Francisco, CA (US)

(72) Inventors: Savon Sampson, Glen Allen, VA (US); Mark Dekovic, Crystal, MN (US); Joe McCracken, Henrico, VA (US)

(21) Appl. No.: 14/665,563

(22) Filed: Mar. 23, 2015

(51) Int. Cl. G06Q 50/22; G06Q 30/06; G06F 19/00; A61F 13/15

(52) U.S. Cl. CPC G06Q 50/22; A61F 13/15; G06Q 30/0631; G06F 19/363; A61F 2013/15121

(57) ABSTRACT

A method for providing dynamic evaluation of healthcare consumable needs by providing patient-specific recommendations for types, sizes, and quantities of healthcare consumable goods is provided. Methods may provide for creation of a patient profile including: providing for selection of a patient height, providing for selection of a patient weight, and providing for selection of at least one patient incontinence parameter. Methods may also include generating a recommendation for one or more types of products for the treatment of incontinence to be associated with the patient profile, generating a recommendation for a size of at least one of the one or more types of products for the treatment of incontinence, and providing for a selection for a selection of a quantity of the one or more types of products for treatment of incontinence.

### TECHNOLOGICAL FIELD

Embodiments of the present invention relate generally to healthcare consumable management solutions and, more particularly, relate to the dynamic evaluation of healthcare consumable needs including providing recommendations for types, quantities, and replenishment of consumables.

### BACKGROUND

Healthcare costs are a hot topic in the media and general population. Both government entities and business entities are focusing on ways to manage healthcare costs to improve affordability and access, and to reduce waste. Thus, in many cases, healthcare related service providers are looking for ways to improve patient care and organizational management by, for example, streamlining their own information management and internal processes. Some of these streamlining efforts have been facilitated or even rely on the use of computers given that there is a push to enable many healthcare management related tasks to be handled by computers. However, the overall success of any healthcare program relies largely on the ease of implementation and the perceived benefit in order to engage a program user and maintain use of the program.

Healthcare programs intended to reduce costs may be of limited success if they are not fully implemented within a healthcare system. Thus it is important for healthcare programs for reducing cost to be user friendly (i.e., easy to use), provide a tangible benefit, and be seamlessly integrated.

### BRIEF SUMMARY

A method, apparatus and computer program product are therefore provided to dynamically evaluate healthcare consumable needs by providing patient-specific recommendations for types, sizes, and quantities of healthcare consumable goods. Accordingly, methods of example embodiments may provide for creation of a patient profile including: providing for selection of a patient height, providing for selection of a patient weight, and providing for selection of at least one patient incontinence parameter. Methods may also include generating a recommendation for one or more types of products for the treatment of incontinence to be associated with the patient profile, generating a recommendation for a size of at least one of the one or more types of products for the treatment of incontinence, and providing for a selection of a quantity of the one or more types of products for treatment of incontinence. The at least one patient incontinence parameter may include one or more of urinary frequency, bowel frequency, or mobility level. Generating a recommendation for one or more types of products for treatment of incontinence may include applying an algorithm to a selected patient height, a selected patient weight, and at least one of a selected at least one patient incontinence parameter, where the recommendation is dependent upon the selected patient height and patient weight. The generated recommendation may further include a quantity of the at least one of the one or more types of products for the treatment of incontinence.

According to some embodiments, generating a recommendation for a quantity of the at least one of the one or more types of products for the treatment of incontinence may include applying an algorithm to at least one of the selected at least one patient incontinence parameters, where the quantity of the one or more types of products is dependent upon the at least one of a selected patient incontinence parameter. Providing for selection of a patient height, providing for selection of a patient weight, and providing for selection of at least one patient incontinence parameter may each be performed through a graphical user interface. Methods may include determining a predicted length of stay of a patient in a healthcare facility and providing for recommendation of a quantity of the one or more types of products for treatment of incontinence in response to the predicted length of stay. Methods may optionally include receiving a selection of a quantity of the one or more types of products for treatment of incontinence and providing for recommendation of a reorder frequency in response to the selected quantity of the one or more types of products for treatment of incontinence and the predicted length of stay.

Embodiments of the present invention may provide an apparatus for dynamically evaluating healthcare consumable needs. The apparatus may include at least one processor and at least one memory including computer program code. The at least one memory and the computer program code, with at least one processor, may cause the apparatus to provide for creation of a patient profile. Providing for creation of a patient profile may include causing the apparatus to provide for selection of a patient height, provide for selection of a patient weight, and provide for selection of at least one patient incontinence parameter. The apparatus may further be caused to generate a recommendation for one or more types of products for treatment of incontinence to be associated with the patient profile, generate a recommendation for a size of at least one of the one or more types of products for the treatment of incontinence, and provide for selection of a quantity of the one or more types of products for treatment of incontinence. The at least one patient incontinence parameter may include one or more of urinary frequency, bowel frequency, or mobility level.

According to some embodiments, causing the apparatus to generate a recommendation for one or more types of products for treatment of incontinence may include causing the apparatus to apply an algorithm to a selected patient height, a selected patient weight, and at least one of a selected at least one patient incontinence parameter, where the recommendation may be dependent upon the selected patient height and the selected patient weight. The apparatus may optionally be caused to generate a recommendation for a quantity of the at least one of the one or more types of products for the treatment of incontinence. Causing the apparatus to generate a recommendation for a quantity of the at least one of the one or more types of products for the treatment of incontinence may include causing the apparatus to apply an algorithm to at least one of the selected at least one patient incontinence parameters, and where the quantity of the one or more types of products is dependent upon the at least one of a selected patient incontinence parameter.

According to some embodiments, causing the apparatus to provide for selection of a patient height, causing the apparatus to provide for selection of a patient weight, and causing the apparatus to provide for selection of at least one patient incontinence parameter may each be provided through a graphical user interface. The apparatus may optionally be caused to determine a predicted length of stay for a patient in a healthcare facility and cause the apparatus to provide for recommendation of a quantity of the one or more types of products for treatment of incontinence in response to the predicted length of stay. The apparatus of some embodiments may be caused to receive a selection of a quantity of the one or more types of products for treatment of incontinence and provide for recommendation of a reorder frequency in response to the selected quantity of the one or more types of products for treatment of incontinence and the predicted length of stay.

Embodiments described herein may provide a computer program product having at least one non-transitory computer-readable storage medium having computer-executable program code instructions stored therein. The computer-executable program code instructions may include program code instructions for creating a patient profile, including program code instructions for providing for selection of a patient height, program code instructions for providing for selection of a patient weight, and program code instructions for providing for selection of at least one patient incontinence parameter. The computer program product may further include program code instructions for generating a recommendation for one or more types of products for treatment of incontinence to be associated with the patient profile, program code instructions for generating a recommendation for a size of at least one of the one or more types of products for treatment of incontinence, and program code instructions for providing for selection of a quantity of the one or more types of products for treatment of incontinence. The at least one patient incontinence parameter may include one or more of a urinary frequency, a bowel frequency, or a mobility level.

According to some embodiments, the program code instructions for generating a recommendation for one or more types of products for treatment of incontinence may include program code instructions for applying an algorithm to a selected patient height, a selected patient weight, and at least one of a selected at least one patient incontinence parameter, where the recommendation is dependent upon the selected patient height and the selected patient weight. The computer program product may optionally include program code instructions for generating a recommendation for a quantity of the at least one of the one or more types of products for treatment of incontinence.

### BRIEF DESCRIPTION OF THE SEVERAL VIEWS OF THE DRAWING(S)

Having thus described embodiments of the invention in general terms, reference will now be made to the accompanying drawings, which are not necessarily drawn to scale, and wherein:

FIG. 1 is a block diagram illustrating a system for the dynamic evaluation of healthcare consumable needs according to an example embodiment of the present invention;

FIG. 2 is a block diagram showing various components that may be included in an apparatus for providing dynamic evaluation of healthcare consumable needs according to an example embodiment of the present invention;

FIG. 3 is a user interface for a system for the dynamic evaluation of healthcare consumable needs according to an example embodiment of the present invention;

FIG. 4 is another user interface for a system for the dynamic evaluation of healthcare consumable needs according to an example embodiment of the present invention;

FIG. 5 is still another user interface for a system for the dynamic evaluation of healthcare consumable needs according to an example embodiment of the present invention;

FIG. 6 is an order screen user interface for a system for the dynamic evaluation of healthcare consumable needs according to an example embodiment of the present invention; and

FIG. 7 is a flowchart of a method for the dynamic evaluation of healthcare consumable needs according to an example embodiment of the present invention;

### DETAILED DESCRIPTION

Embodiments of the present invention will now be described more fully hereinafter with reference to the accompanying drawings, in which some, but not all embodiments of the invention are shown. Indeed, embodiments of the invention may be embodied in many different forms and should not be construed as limited to the embodiments set forth herein; rather, these embodiments are provided so that this disclosure will satisfy applicable legal requirements. Like reference numerals refer to like elements throughout.

As indicated above, embodiments of the present invention are aimed at providing a mechanism for dynamically evaluating health care consumable needs including providing for recommendations of types, quantities, and replenishment of consumables. Some example embodiments may be used to support the provision of an incontinence spend evaluation tool used in a healthcare environment. The incontinence spend evaluation tool may be implemented to determine the needs of a particular patient, recommend one or more types of products for the treatment of incontinence based on a profile of the patient, recommend a quantity for the one or more types of products based on the profile of the patient, and recommend one or more reorder/replenishment points.

Embodiments of the present invention may be implemented in a healthcare environment with a plurality of patients, where the recommendations for products, quantities, and replenishment, may be performed on a patient-by-patient analysis of the patients within a healthcare facility. According to some embodiments, algorithms may be used with patient parameters as inputs to identify the types, quantities, and replenishment schedule for products related to the patient's specific issues. Some example embodiments may further facilitate an ordering/reordering process of the identified and selected products. While the primary embodiments described herein may be implemented in a healthcare facility with a plurality of patients, embodiments may optionally be implemented for personal use of individual patients, for example in an in-home care environment.

Healthcare facilities may serve dozens or hundreds of patients on a routine basis, and each of these patients may have differing needs for care based on their condition. Each patient may require certain consumables during their stay in a healthcare facility such that a healthcare facility may need to maintain an inventory of consumable goods that satisfies the needs of a plurality of potential patients, and has sufficient quantities to satisfy the needs of a plurality of potential patients requiring the same consumables. Inventory planning for a healthcare facility can be difficult and may lead to large quantities of various consumables being stored in inventory in anticipation of potential need. These consumables, some of which may have expiration dates, may sit in inventory for weeks, months, or years, or the consumables may be consumed very quickly, in dependence of the type of consumables needed by a plurality of patients at any given time. Further, these consumables may be expensive to purchase, and the storage requirements of space within a facility may be expensive to maintain. Thus, accurate inventory control is important to help reduce inventory carrying costs.

While accurate inventory control can reduce inventory carrying costs, healthcare facilities may not be able to carry a full complement of inventory to adequately supply the facility due to cost and space concerns. Embodiments of the present invention address these issues through the dynamic evaluation of healthcare consumable needs. This dynamic evaluation can facilitate just-in-time delivery of the necessary consumables such that inventory carrying costs are greatly reduced and patients receive the consumables they need in a timely manner. Example embodiments described herein help a user and/or a healthcare professional to identify the consumable needs of a patient, identify the quantities of consumable needs of a patient, and identify the replenishment schedule of consumables needed for the patient.

As noted above, an example of consumables that may vary in quantity and type from patient to patient includes products for the treatment of incontinence issues. Incontinence is a common problem affecting millions of people in the United States, and is particularly prevalent among the elderly and is relatively common among women. While there are medications and surgeries that can help reduce or eliminate incontinence issues, there exists a need to treat the symptoms of incontinence issues that may be temporary or for which no other solution is practical. The treatment of the symptoms of incontinence can include the use of absorbent materials such as absorbent undergarments or absorbent undergarment liners or pads.

Products for the treatment of incontinence can come in a variety of shapes and sizes, where the size and shape may be dependent upon the height, weight, and gender of the user. The products may also depend upon the degree of mobility of a patient. Further, the quantity of products for the treatment of incontinence will vary by individual based on their urinary and bowel frequency. Thus, it is difficult for a healthcare facility to adequately manage inventory for a population of future potential patients when the gender, height, weight, and mobility of the potential patients, together with their urinary/bowel frequency, is an unknown factor.

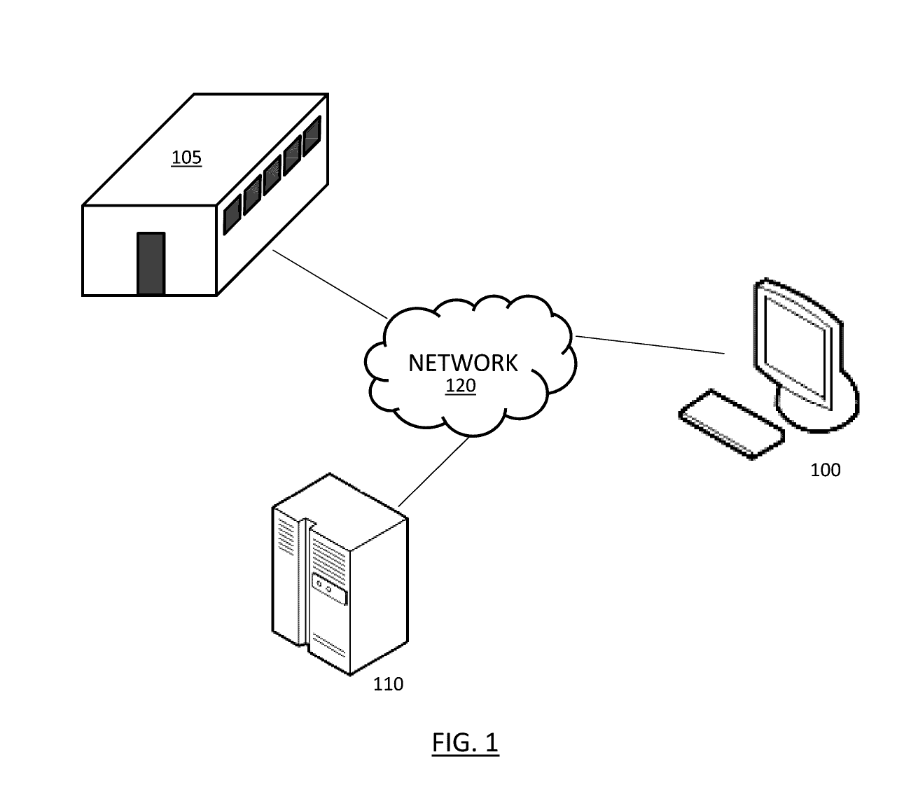

An example embodiment will now be described in reference to FIG. 1, which illustrates an example system in which an embodiment of the present invention may be implemented. Of note, the example of FIG. 1 is provided to illustrate several, but not all examples of devices and system architectures that may employ example embodiments. Thus, some examples may include more or fewer components than those which are shown in FIG. 1. As depicted in FIG. 1, a system according to an example embodiment may include one or more user terminals 100, one or more inventory facilities 105, one or more servers 110, each in communication with a network 120.

Embodiments may include other network entities from which data may be received from or transmitted to, as will be described further below. Each of the components of the system may be in electronic communication with, for example, one another over the same or different wireless or wired networks (e.g., network 120) including, for example, a wired or wireless Personal Area Network (PAN), Local Area Network (LAN), Metropolitan Area Network (MAN), Wide Area Network (WAN), or the like. Additionally, while FIG. 1 illustrates the various system entities as separate, standalone entities, the various embodiments are not limited to this particular architecture. Further, while the illustrated embodiment of FIG. 1 depicts a user terminal 100 that is separate from the server 110, example embodiments may include where the user terminal 100 and server 110 are embodied in a single entity. The user terminal 100 may be located within a healthcare facility for use by a healthcare professional. Optionally, a user terminal may be located with a patient at home implementations in which the user is the patient in a home care setting.

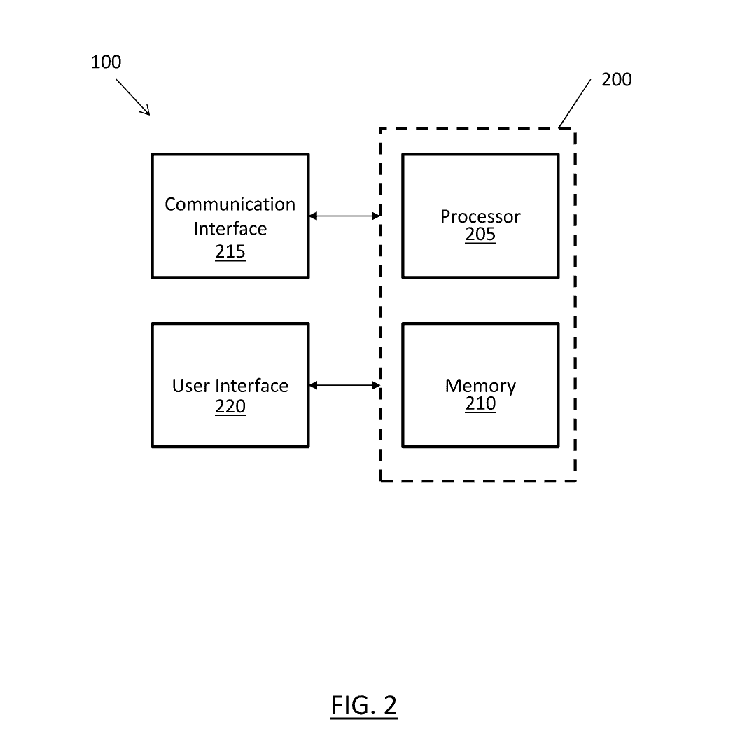

Although the user terminal 10 may be configured in various manners, one example of a user terminal that may benefit from embodiments of the invention is depicted in the block diagram of FIG. 2. A user terminal may be embodied as any one of a variety of computing devices, such as a portable digital assistant (PDA), cellular telephone or smart-phone, all types of computers (e.g., laptops, tablets, mobile computers, desktop computers, etc.), or any device which may facilitate the user interface and communications described wherein with respect to the user terminal 100.

FIG. 2 illustrates a block diagram of a user terminal 100 in accordance with some example embodiments that may be capable of functioning in a health information infrastructure and may be embodied by one or more servers, computer workstations, desktop or laptop computers or the like. As described below, the user terminal 100 may be configured to implement and/or otherwise support implementation of various example embodiments. However, it should be noted that the components, devices or elements illustrated in and described with respect to FIG. 2 below may not be mandatory and thus some may be omitted in certain embodiments. Additionally, some embodiments may include further or different components, devices or elements beyond those illustrated in and described with respect to FIG. 2.

The user terminal 100 may include or otherwise be in communication with processing circuitry 200 that is configurable to perform actions in accordance with one or more example embodiments disclosed herein. In this regard, the processing circuitry may be configured to perform and/or control performance of one or more functionalities of the user terminal 100 in accordance with various example embodiments, and thus may provide means for performing functionalities of the user terminal 100. The processing circuitry may be configured to perform data processing, application execution and/or other processing and management services according to one or more example embodiments.

In some example embodiments, the processing circuitry 200 may include a processor 205 and, in some embodiments may further include memory 210. The processing circuitry may be in communication with or otherwise control a communication interface 215 and, in some embodiments, a user interface 220. As such, the processing circuitry may be embodied as a circuit chip (e.g., an integrated circuit chip) configured (e.g., with hardware, software or a combination of hardware and software) to perform operations described herein.

The processor 205 may be embodied in a number of different ways. For example, the processor may be embodied as various processing means such as one or more of a microprocessor or other processing element, a coprocessor, a controller or various other computing or processing devices including integrated circuits such as, for example, an ASIC (application specific integrated circuit), an FPGA (field programmable gate array), or the like. Although illustrated as a single processor, it will be appreciated that the processor may comprise a plurality of processors. The plurality of processors may be in operative communication with each other and may be collectively configured to perform one or more functionalities of the user terminal 100 as described herein. The plurality of processors may be embodied on a single computing device or distributed across a plurality of computing devices collectively configured to function as the user terminal. In some example embodiments, the processor 205 may be configured to execute instructions stored in the memory 210 or otherwise accessible to the processor. As such, whether configured by hardware or by a combination of hardware and software, the processor 205 may represent an entity (e.g., physically embodied in circuitry—in the form of processing circuitry 200) capable of performing operations according to embodiments of the present invention while configured accordingly. Thus, for example, when the processor 205 is embodied as an ASIC, FPGA or the like, the processor may be specifically configured hardware for conducting the operations described herein. Alternatively, as another example, when the processor 205 is embodied as an executor of software instructions, the instructions may specifically configure the processor to perform one or more operations described herein.

In some example embodiments, the memory 210 may include one or more non-transitory memory devices such as, for example, volatile and/or non-volatile memory that may be either fixed or removable. In this regard, the memory may comprise a non-transitory computer-readable storage medium. It will be appreciated that while the memory is illustrated as a single memory, the memory may comprise a plurality of memories. The plurality of memories may be embodied on a single computing device or may be distributed across a plurality of computing devices collectively configured to function as the user terminal 100. The memory 210 may be configured to store information, data, applications, instructions and/or the like for enabling the computing device to carry out various functions in accordance with one or more example embodiments. For example, the memory may be configured to buffer input data for processing by the processor 205. Additionally or alternatively, the memory 210 may be configured to store instructions for execution by the processor. As yet another alternative, the memory may include one or more databases that may store a variety of files, contents or data sets. Among the contents of the memory, applications may be stored for execution by the processor in order to carry out the functionality associated with each respective application. In some cases, the memory 210 may be in communication with one or more of the processor 205, user interface 220, or communication interface 215 via a bus or buses for passing information among components of the computing device.

The user interface 220 may be in communication with the processing circuitry 200 to receive an indication of a user input at the user interface and/or to provide an audible, visual, mechanical or other output to the user. As such, the user interface 220 may include, for example, a keyboard, a mouse, a joystick, a display, a touch screen display, a microphone, a speaker, a Light Emitting Diode (LED), a lighting device, an electronic sensor for capturing human body movements, and/or other input/output mechanisms. In embodiments in which the user terminal is implemented on a server, aspects of the user interface may be limited, or the user interface may even be eliminated. For example, the computing device may act as a server or host device, with a user interface provided by a client application.

The communication interface 215 may include one or more interface mechanisms for enabling communication with other devices and/or networks, such as with the healthcare facilities. In this regard, communication with the healthcare facilities includes communication with one or more computing devices of the respective healthcare facilities. In some cases, the communication interface may be any means such as a device or circuitry embodied in either hardware, or a combination of hardware and software that is configured to receive and/or transmit data from/to a network and/or any other device or module in communication with the processing circuitry 200. By way of example, the communication interface may be configured to enable the user terminal 100 to communicate with the healthcare facilities via a wireless network, such as a wireless local area network (WLAN), cellular network, and/or the like. Additionally or alternatively, the communication interface may be configured to enable the computing device to communicate with the healthcare facilities via a wire-line network. In some example embodiments, the communication interface 215 may be configured to enable communication between the user terminal and one or more healthcare facilities via the internet. Accordingly, the communication interface 220 may, for example, include an antenna (or multiple antennas) and supporting hardware and/or software for enabling communications with a wireless communication network (e.g., a wireless local area network, cellular network, and/or the like) and/or a communication modem or other hardware/software for supporting communication via cable, digital subscriber line (DSL), universal serial bus (USB), Ethernet or other methods.

Embodiments of the user terminal 100 may include an inventory management computer system that is application specific for monitoring, controlling, and facilitating the replenishment of inventory. The inventory management system of example embodiments may be uniquely configured for healthcare products such as products related to incontinence treatment. Such embodiments may interface with healthcare information systems and supplier ordering systems through, for example, communications interface 215. The healthcare information system may provide patient identification or limited patient identifiers (which may be made anonymous for security concerns, privacy concerns, or HIPAA compliance, for example). The supplier ordering system may provide an interface through which example embodiments of the inventory management system may provide orders and replenishment schedules for delivery of inventory items on an as-needed basis.

Having now described user terminal 100 configured to implement and/or support implementation of various example embodiments, features of several example embodiments will now be described. It will be appreciated that the following features are non-limiting examples of features provided by some example embodiments. Further, it will be appreciated that embodiments are contemplated within the scope of disclosure that implement various subsets or combinations of the features further described herein. Accordingly, it will be appreciated that some example embodiments may omit one or more of the following features and/or implement variations of one or more of the following features.

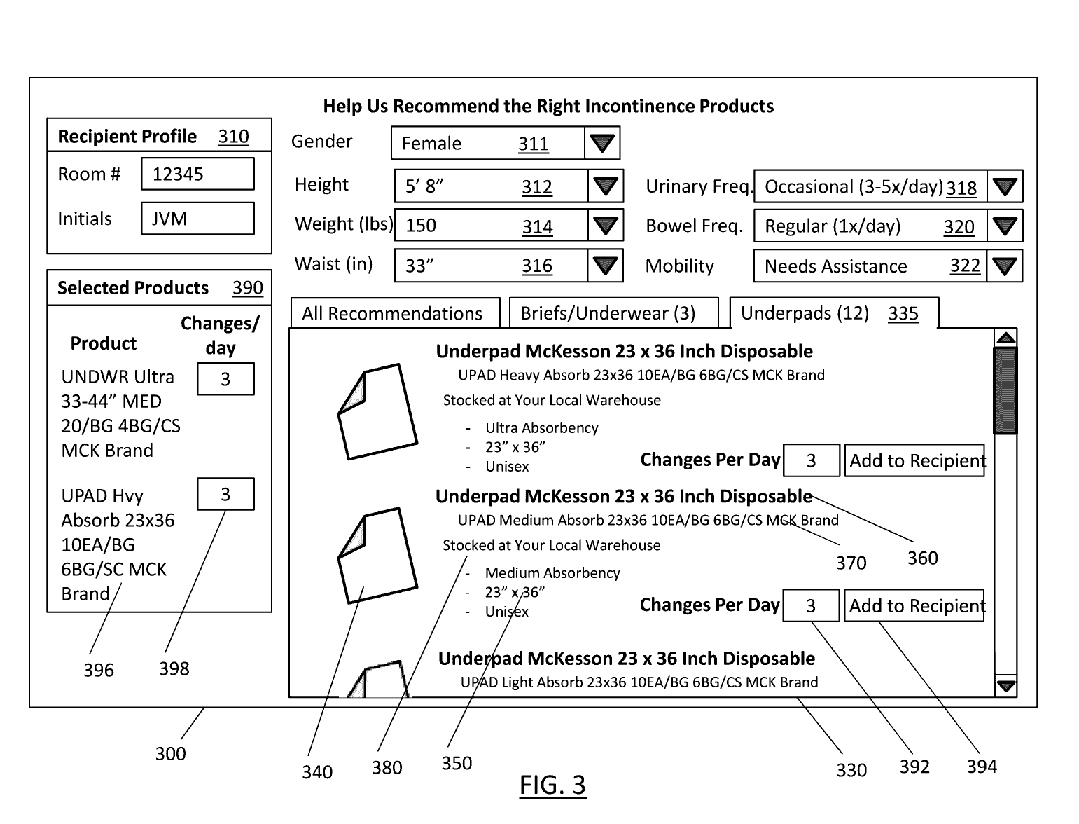

An example embodiment of the invention will now be described with reference to FIG. 3 which depicts an example embodiment of a user interface for a system for the dynamic evaluation of healthcare consumable needs. The depicted embodiment includes a user interface 300 which may be presented, for example, on a display of a user terminal 100. The user interface 300 may include a recipient profile section 310 that is used to identify a patient by location and identity. In the instant embodiment, the patient is identified by “Room #” and the patients “Initials”. This identification may enable access to an established patient profile at a later time without requiring the user to enter in detailed information about the patient. The user interface 300 may also include a plurality of patient parameters that can be entered by a user. For example, the gender 311, the height 312, weight 314, and waist measure 316, of a patient may be entered into the user interface via dropdown boxes, text boxes, radio buttons, or any manner by which the patient information may be communicated through the user interface. Additional parameters may be available for selection by a user, including a “Urinary Frequency” 318 which may be the number of times a user urinates during a specific period of time, such as a 24-hour day. Optionally, the frequency may be the number of incontinence accidents that occur during a specific period of time. The “Bowel Frequency” 320 may be similarly employed to specify a patient's bowel habits, either by general daily frequency or by incontinence issue daily frequency. An additional patient parameter 322 may be included for the “Mobility” of the patient. This may specify if the patient is self-sufficient in using the bathroom or if they require some degree of assistance.

Each of the aforementioned parameters (312-322) may be used to provide recommendations for products for the treatment of incontinence. Once the parameters 312-322 have been entered, recommendations for one or more types of products for treatment of incontinence may be generated. It is noted that a recommendation may be provided without all of the parameters being selected. For example, a patient's waist measurement may not be readily available if they are bed-ridden, and the parameter may be left blank. A recommendation can be generated with fewer than all of the parameters specified; however, the recommendation may be more accurate with a greater number of parameters specified. Further description of the algorithms employed is provided below.

FIG. 3 illustrates a number of recommendations provided in window 330. The recommendations may be broken down by category into a plurality of tabs, such as tab 335 that is used to depict “Underpad” products that are recommended for the patient. The parenthetical number adjacent to the tab title may indicate the number of recommendations under that category. The tab labeled “Briefs/Underwear” may include undergarment-related products and may be selected by a user to display only those undergarment products in the window 330. The recommendations may be arranged in a manner that provides the most relevant recommendations or the most heavily weighted recommendations at the top of the list of recommendations. For example, the recommendations may include underpads as shown in FIG. 3, and based upon a selected parameter, such as urinary frequency 318, the “heavy absorbency” underpad may be the strongest recommendation. The underpad recommendations may recommend, but less strongly, the medium absorbency underpad, which is situated below the heavy absorbency underpad recommendation, indicating that it is not as strongly recommended.

The user may review the recommended products in window 330 including one or more of the illustrated images 340, product features 350, product name 360, and brief product description 370. The recommended products may also include an indicator of where the products may be found to inform a user of the availability of the products. Products stocked locally at a local warehouse may be more readily available than products stocked at a regional warehouse, for example. This may affect the lead-time for receiving ordered products, and may affect a user's selection based on immediacy of a product need.

A user may add products to the “Selected Products” 390 to be added to an order for the patient identified in the “Recipient Profile” 310, as will be described further below. A user may select a quantity 392 of a selected product and provide an input (e.g., via a touchscreen, a mouse/cursor, etc.) to the “Add to Recipient” button 394 to add the identified quantity to the “Selected Products” list 390. It is noted that the quantity identified in the user interface 300 as “Changes Per Day” may not reflect an individual item quantity. For example, an underpad product for a male may be a 2-piece set such that two pieces are included for each “Change Per Day”. The “Change Per Day” may be a more accurate identifier to enable a user to appropriately select the number of products needed for a particular patient without requiring the user to separately calculate individual units versus changes per day. The quantity 392 may be pre-populated based on the patient parameters entered, such as the urinary frequency and/or the bowel frequency. While a user may manually alter the prepopulated quantities 392, this pre-population enables a user to see what is recommended based on the entered patient information by the algorithms used by the incontinence spend evaluation tool.

Once the items are selected through the “Add to Recipient” button 394, the product 396 and quantity 398 are added to the selected products window 390. This enables a user to see which products have already been selected to help avoid duplicate ordering or accidental failure to order a particular product. The user may build a list of selected items for a particular patient until all incontinence-related products for a particular patient have been properly selected. The user may then add a new recipient or edit an existing recipient. Once all recipients or patients are properly entered and incontinence products selected for each of them, the user may bring up an ordering interface.

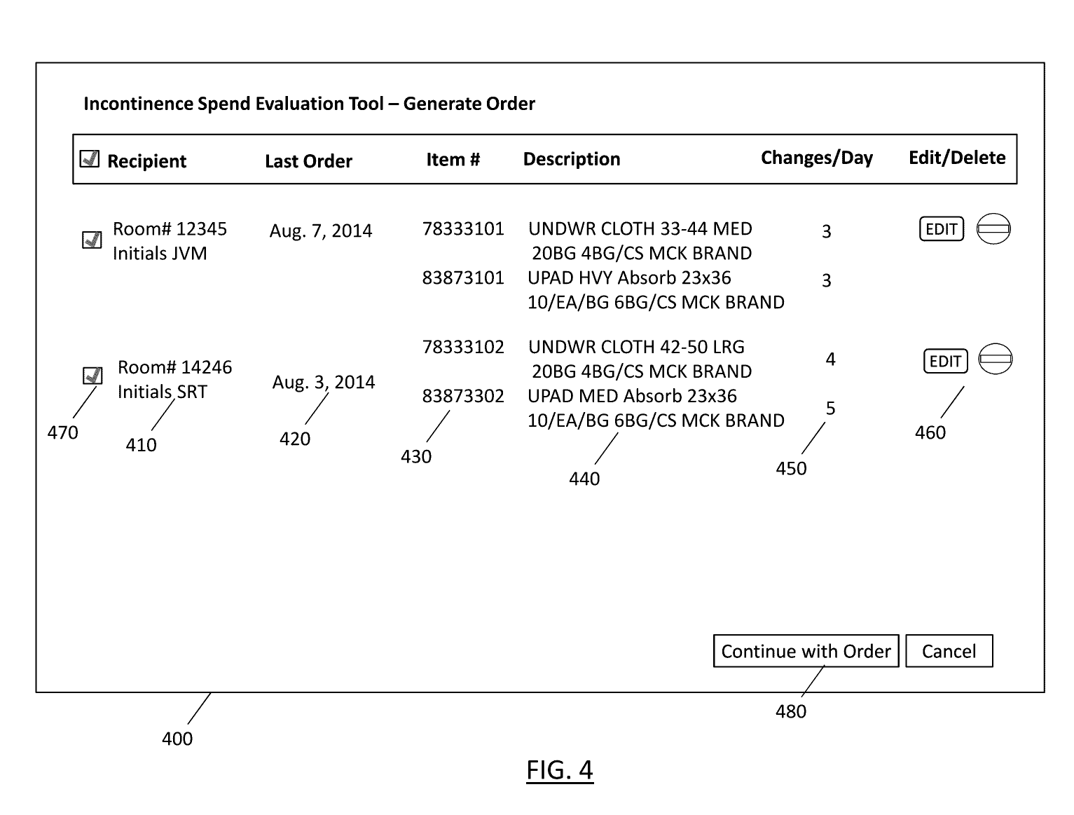

FIG. 4 illustrates an example embodiment of the ordering interface 400 which may be presented, for example, on a display of user terminal 100. The depicted ordering interface illustrates the patient recipient 410 identified by room number and initials. While the depicted embodiments identify patients by room number and initials, it is appreciated that any number of identifiers may be used to uniquely identify patients, such as patient identification numbers, names with birth dates, social security numbers, etc. The depicted ordering interface further includes a last order date 410 such that a user can readily identify the last time an order was placed for a particular patient. The item number 430 and item descriptions 440 may be provided, together with the quantity 450 selected. Each individual recipient's order can be edited or deleted as needed through selection of an icon in the last column 460. A radio button 470 or other selection means may be located next to each recipient in order to either include or exclude the particular patient from an order. A “Continue with Order” 480 button may be present to indicate to the user to advance to the next step of the order process.

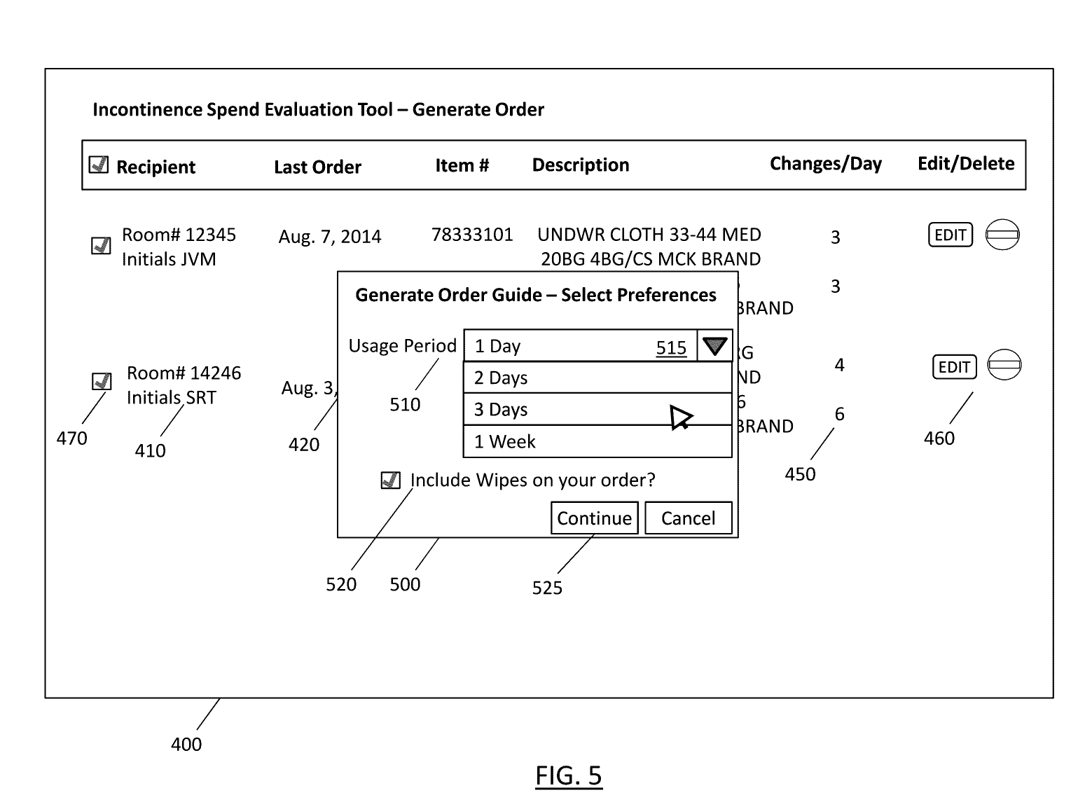

FIG. 5 illustrates an example embodiment of an interstitial order screen that may be presented in response to a user selecting the “Continue with Order” 480 button of the ordering interface 400. The interstitial order screen 500 may request a user to enter additional information about the order, such as the usage period 510 of the ordered products. The usage period 510 may be selected, for example, through drop-down menu 515. The usage period defines the amount of time the products ordered are intended to cover. For example, the selections may include any number of predefined time periods, such as one, two, three, or more days, one or more weeks, one or more months, etc. Upon selection, embodiments of the present invention may store the order information and the period of time over which the ordered products are intended to be used. This information may be beneficial to identify products requiring reorder and replenishment frequency, as described further below.

The interstitial screen 500 may also include an option to add products to the order. For example, wipes are a unisex, one-size-fits-all type of product such that wipes can be a generic “add-on” to any order if selected, for example, through radio button 520. Upon selecting the usage period and any available/desired add-on items, the user may click the “continue” button 525 to continue with the order.

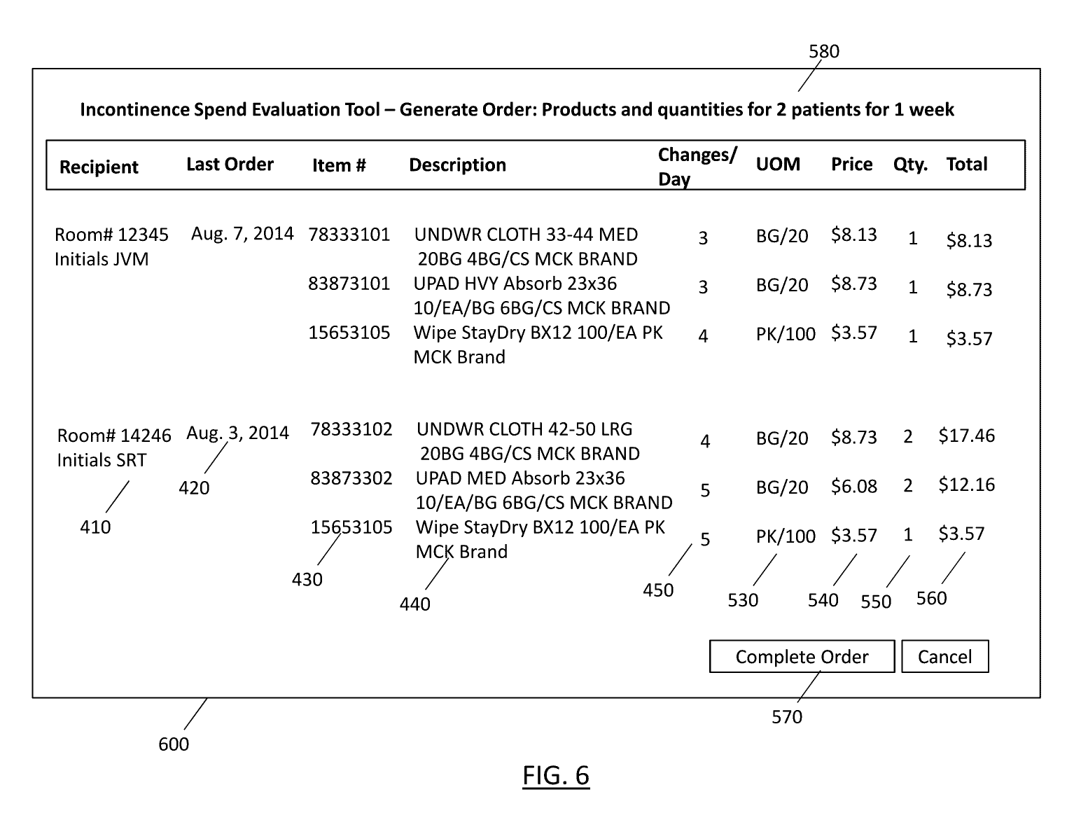

FIG. 6 illustrates the order guide including additional information determined through the selected changes per day and the selected length of time. The header 580 identifies that the order is for two recipients or patients, and the selected usage period is one week. The first line-item for item 78333101 indicates that 3 changes per day are required. As such, for one week, 21 units of that item will be required. The incontinence spend evaluation tool illustrated calculates this number, and references the “unit of measure” or “UOM” of a package of the products. For item 78333101, the package quantity is 20. While 21 may be estimated to be needed, the evaluation tool may include an algorithm with a rounding factor that will round down when close to the target quantities. In the instant embodiment, two packages of the product would include 40 products, 95% more than is required. Thus, the evaluation tool may calculate that it is more advantageous to order a package of 20 rather than two packages of 20. The algorithm employing a rounding factor may consider the cost of the item, the resultant over-supply if the package quantity is incrementally increased, the cost of the item, the risks associated with under-supply of the item, and the prior order quantities, as described further below. The price, quantity, and total price for the order are also illustrated.

For patient SRT in room number 14246, the patient may need 4 changes/day of item 78333102 which would equate to 28 units for a week. While two packages include 40 units, the evaluation tool algorithm may determine that the 8 units beyond what a single package contains merits the ordering of a second package to ensure enough units are on hand for the selected usage period. A user may review the order guide user interface 600 of FIG. 6 and may adjust quantities manually based on personal knowledge of a patient, or based upon historical knowledge of particular product consumption. However, generally the incontinence spend evaluation tool will provide accurate estimates of quantities based on the input patient parameters.

Upon verifying the order information from the user interface 600 of FIG. 6, a user may complete the order with button 570, whereupon the order is transmitted to the facility to fulfill the order. Optionally, particularly in larger healthcare facilities, completed orders may be compiled for sending periodically, such as twice daily, to a fulfillment facility in order to combine items for shipping and reduce costs.

According to some embodiments, upon completion of an order, a replenishment reminder may be generated. The replenishment reminder may be based on, for example, the usage period identified in the interstitial window of FIG. 5. This replenishment reminder may include an indication of the types and quantities of products ordered for a patient. The replenishment reminder may further consider an estimated length of stay of a patient derived from a patient's electronic medical record, through an analysis of prior patient stays, or through an analysis of patients having similar dispositions. Further, the replenishment reminder may consider excess quantities of items that were ordered above the anticipated need of a patient due to package quantities, as noted above with regard to the algorithm used for generating a rounding factor. For example, in the example embodiment illustrated in FIG. 6, the patient in Room #14246 had a total quantity of 40 units of item #78333102, when the patient required only 28 units for the week. Thus, the replenishment order may recognize the excess quantity of 12 units from the prior order, and include in the replenishment order a quantity of only 20 units, which together with the 12 excess units, cover the anticipated requirement of 28 units for the week. The following replenishment order may then revise the order quantity to two packages of 20 units with the understanding that the excess capacity of the original order was mostly consumed.

As noted above, according to example embodiments described herein, recommendations for sizes and types of products for the treatment of incontinence may be generated based upon the parameters entered with respect to a patient. The recommendations may be generated by the application of an algorithm to the patient parameters. For example, the parameters including patient gender, patient height, patient weight, and patient waist, may each be variables used as inputs to an algorithm to identify the types of incontinence treatment products for the patient. The size of pads or undergarments may be determined based on these parameters, and further, the type of pad or undergarment may be determined based on these parameters. One such example may include a patient that is morbidly obese that may not be a candidate for absorbent undergarments. Instead, the algorithm may determine, based on the patient height, weight, and waist measure, that the most suitable product for the patient is an absorbent pad of a particular size. Accordingly, the algorithm may present this recommendation to a user through the user interface as illustrated in FIG. 3.

The patient mobility parameter may further be used by the algorithm to generate recommendations for products to treat incontinence for a patient. For example, a patient may have partial or low mobility, where they require assistance to use the bathroom. A facility may be equipped to help the individual during the day; however, over night the patient may not be able to leave their bed. In such a case, the algorithm may identify the mobility issue through the mobility parameter and recommend absorbent undergarments for day-use and absorbent pads for evening use.

Additional parameters, such as the urinary frequency or bowel frequency may be used as inputs by one or more algorithms to determine a quantity of products for the treatment of incontinence. In an example, a user may enter a urinary incontinence frequency of five times per day. An algorithm may interpret the urinary incontinence issue to require the change of a specific product for the treatment of incontinence, and the product may differ between a male and a female patient. The algorithm may thus generate a number of changes per day of a particular product that has been determined by the incontinence spend evaluation tool as recommended for the patient. The recommended number of changes per day may be indicated by the changes-per-day quantity 392 as shown in FIG. 3.

Algorithms may optionally correlate product types according to various other patient parameters from the patient profile. For example, incontinence frequency may be used to provide a recommendation on a type of absorbent pad or undergarment. Occasional incontinence may correlate to light absorbency pads or garments, frequent incontinence may correlate to regular absorbency, and persistent incontinence may correlate to ultra or heavy absorbency pads or garments. According to some embodiments, mobility parameters may be used to establish recommendations for types of products. A patient requiring no mobility assistance may be offered or recommended pant liners, mesh pants, bladder control pads, protective underwear, underpads, or the like. A patient who requires assistance may be offered protective underwear and underpads, while garments or products requiring more patient dexterity may not be recommended. A patient with limited mobility may be recommended briefs and underpads, while a patient who is bed ridden or has no mobility may be offered pant liners, mesh pants, briefs, and underpads. Optionally, the types of products offered or recommended may be established based on care giver assistance needs, such as a patient requiring mobility assistance who also weighs in excess of 300 pounds may not be recommended products that would be difficult for a care giver to facilitate application of to the patient. Other patient parameters in a patient profile may exclude certain products from recommendations. For example, if a patient has frequent bowel incontinence, the patient may not receive a recommendation for pant liners, mesh pants, or bladder control pads.

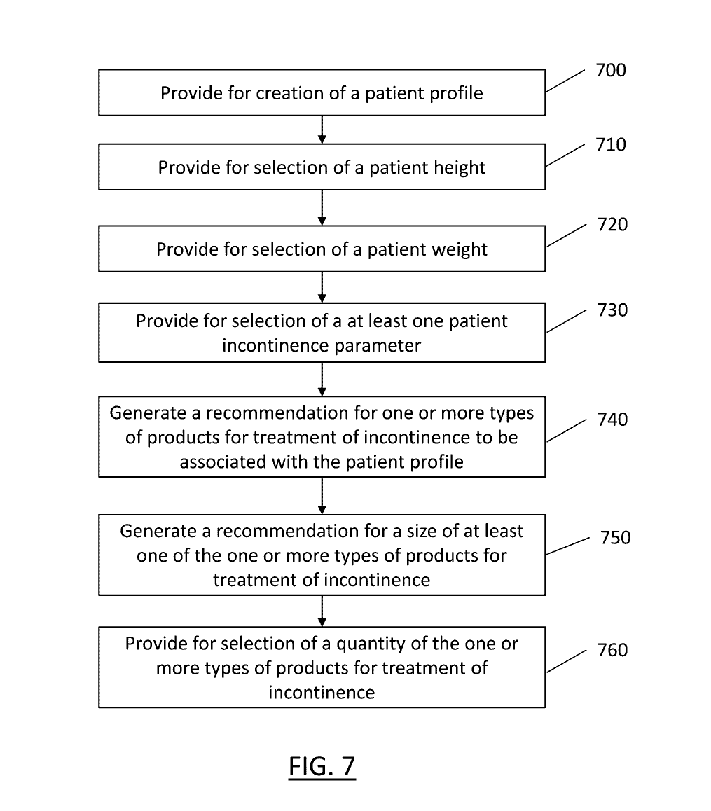

Embodiments of the present invention may be practiced using an apparatus such as the one depicted in FIG. 2 within the overall system depicted in FIG. 1. However, it should be appreciated that some embodiments may be practiced in connection with a computer program product for performing embodiments of the present invention. FIG. 7 is a flowchart of a method, apparatus, and program product according to exemplary embodiments of the invention. Each block of the flowchart of FIG. 7, and combinations of blocks in the flowchart, may be implemented by various means, such as hardware, firmware, processor, circuitry and/or another device associated with execution of software including one or more computer program instructions. Thus, for example, one or more of the procedures described above may be embodied by computer program instructions, which may embody the procedures described above and may be stored by a storage device (e.g., memory 210) and executed by processing circuitry (e.g., processor 205). The operations of FIG. 7 may define operations for the execution of an algorithm for dynamic evaluation of consumable consumption and the evaluation of consumable needs. Furthermore, it should be noted that any of the operations of FIG. 7 may be repeated in some embodiments in order to provide recommendations for types, quantities, and replenishment of consumables.

As will be appreciated, any such stored computer program instructions may be loaded onto a computer or other programmable apparatus (i.e., hardware) to produce a machine, such that the instructions which execute on the computer or other programmable apparatus implement the functions specified in the flowchart block(s). These computer program instructions may also be stored in a non-transitory computer-readable medium comprising memory that may direct a computer or other programmable apparatus to function in a particular manner, such that the instructions stored in the computer-readable memory produce an article of manufacture including instructions to implement the function specified in the flowchart block(s). The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart block(s).

In this regard, a method according to one example embodiment of the invention, as shown in FIG. 7, may include providing for creation of a patient profile at 700. The patient profile may be created by providing for selection of a patient height at 710, providing for selection of a patient weight at 720, and providing for selection of at least one incontinence parameter at 730. The patient incontinence parameter may be, for example, a urinary frequency, a bowel frequency, a mobility level, or a combination thereof. A recommendation for one or more types of products for the treatment of incontinence may be generated at 740 for association with the patient profile. A recommendation for a size of the at least one of the one or more types of products for the treatment of incontinence may be generated at 750. According to some embodiments, the method may include providing for selection of a quantity of the one or more types of products for treatment of incontinence as shown at 760.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. An apparatus for comprising at least one processor and at least one memory including computer program code, the at least one memory and the computer program code configured to, with the at least one processor, cause the apparatus to at least perform:
provide for creation of a patient profile, wherein causing the apparatus to provide for creation of a patient profile comprises causing the apparatus to:
provide for selection of a patient height;
provide for selection of a patient weight; and
provide for selection of at least one patient incontinence parameter;
generate a recommendation for one or more types of products for treatment of incontinence to be associated with the patient profile;
generate a recommendation for a size of at least one of the one or more types of products for treatment of incontinence; and
provide for selection of a quantity of the one or more types of products for treatment of incontinence.

2. The apparatus of claim 1, wherein the at least one patient incontinence parameter comprises one or more of a urinary frequency, a bowel frequency, or a mobility level.

3. The apparatus of claim 2, wherein causing the apparatus to generate a recommendation for one or more types of products for treatment of incontinence comprises causing the apparatus to apply an algorithm to a selected patient height, a selected patient weight, and at least one of a selected at least one patient incontinence parameters, wherein the recommendation is dependent upon the selected patient height and the selected patient weight.

4. The apparatus of claim 3, further comprising causing the apparatus to:
generate a recommendation for a quantity of the at least one of the one or more types of products for treatment of incontinence.

5. The apparatus of claim 4, wherein causing the apparatus to generate a recommendation for a quantity of the at least one of the one or more types of products for treatment of incontinence comprises causing the apparatus to apply an algorithm to at least one of the selected at least one patient incontinence parameters, and wherein the quantity of the one or more types of products is dependent upon the at least one of a selected patient incontinence parameter.

6. The apparatus of claim 1, wherein causing the apparatus to provide for selection of a patient height, causing the apparatus to provide for selection of a patient weight, and causing the apparatus to provide for selection of at least one patient incontinence parameter, are each provided through a graphical user interface.

7. The apparatus of claim 1, further comprising causing the apparatus to determine a predicted length of stay for a patient in a healthcare facility and causing the apparatus to provide for recommendation of a quantity of the one or more types of products for treatment of incontinence in response to the predicted length of stay for the patient.

8. The apparatus of claim 7, further comprising causing the apparatus to:
receive a selection of a quantity of the one or more types of products for treatment of incontinence; and
provide for recommendation of a reorder frequency in response to the selected quantity of the one or more types of products for treatment of incontinence and the predicted length of stay.

9. A method for providing a user interface with a system for the dynamic evaluation of healthcare consumable needs, the method comprising:
providing for creation of a patient profile by a processor of the system, wherein creation of a patient profile comprises:
providing for selection within a user interface of a patient height;
providing for selection within the user interface of a patient weight; and
providing for selection within the user interface of at least one patient incontinence parameter;
generating, by the system, a recommendation for one or more types of products for treatment of incontinence to be associated with the patient profile;
generating, by the system, a recommendation for a size of at least one of the one or more types of products for treatment of incontinence; and
providing for selection within the user interface of a quantity of the one or more types of products for treatment of incontinence.

10. The method of claim 9, wherein the at least one patient incontinence parameter comprises one or more of a urinary frequency, a bowel frequency, or a mobility level.

11. The method of claim 10, wherein generating a recommendation for one or more types of products for treatment of incontinence comprises applying an algorithm to a selected patient height, a selected patient weight, and at least one of a selected at least one patient incontinence parameters, wherein the recommendation is dependent upon the selected patient height and the selected patient weight.

12. The method of claim 11, further comprising:
generating a recommendation for a quantity of the at least one of the one or more types of products for treatment of incontinence.

13. The method of claim 12, wherein generating a recommendation for a quantity of the at least one of the one or more types of products for treatment of incontinence comprises applying an algorithm to at least one of the selected at least one patient incontinence parameters, and wherein the quantity of the one or more types of products is dependent upon the at least one of a selected patient incontinence parameter.

14. The method of claim 9, wherein providing for selection of a patient height,
providing for selection of a patient weight, and providing for selection of at least one patient incontinence parameter, are each provided through a graphical user interface.

15. The method of claim 9, further comprising determining a predicted length of stay for a patient in a healthcare facility and providing for recommendation of a quantity of the one or more types of products for treatment of incontinence in response to the predicted length of stay for the patient.

16. The method of claim 15, further comprising:
receiving a selection of a quantity of the one or more types of products for treatment of incontinence; and
providing for recommendation of a reorder frequency in response to the selected quantity of the one or more types of products for treatment of incontinence and the predicted length of stay.

17. A computer program product comprising at least one non-transitory computer-readable storage medium having computer-executable program code instructions stored therein, the computer-executable program code instructions comprising:
program code instructions for providing for creation of a patient profile by a processor, wherein creation of a patient profile comprises:
program code instructions for providing for selection of a patient height;
program code instructions for providing for selection of a patient weight; and
program code instructions for providing for selection of at least one patient incontinence parameter;
program code instructions for generating a recommendation for one or more types of products for treatment of incontinence to be associated with the patient profile;
program code instructions for generating a recommendation for a size of at least one of the one or more types of products for treatment of incontinence; and
program code instructions for providing for selection of a quantity of the one or more types of products for treatment of incontinence.

18. The computer program product of claim 17, wherein the at least one patient incontinence parameter comprises one or more of a urinary frequency, a bowel frequency, or a mobility level.

19. The computer program product of claim 18, wherein the program code instructions for generating a recommendation for one or more types of products for treatment of incontinence comprises program code instructions for applying an algorithm to a selected patient height, a selected patient weight, and at least one of a selected at least one patient incontinence parameters, wherein the recommendation is dependent upon the selected patient height and the selected patient weight.

20. The computer program product of claim 19, further comprising:
program code instructions for generating a recommendation for a quantity of the at least one of the one or more types of products for treatment of incontinence.
